(19) United States
(12) Patent Application Publication
(10) Pub. No.: US 2005/0020490 A1
(43) Pub. Date: Jan. 27, 2005

(54) A Method of Producing an Economical and Ecologically Sound Natural Immunobiotic Extract for Use as a Health Management Instrument and a Replacement for Growth Promotion Antibiotics in Livestock and Companion Animals.

(75) Inventors: Philip Anthony Courie Jr., Florence, KY (US); Shane Patelakis, Cornwall (CA); Amy Jo Miles, Edgewood, KY (US)

Correspondence Address: Amy Miles;Biologics International, Incorporated, 8054 Production Drive, Florence, KY, 41042, US

(73) Assignee: PROGRESSIVE BIOACTIVES INCORPORATED, Cornwall (CA)

(21) Appl. No.: 10/711,980

(22) Filed: Oct. 18, 2004

(51) Int. Cl. A61K 38/16; A61K 31/715; C07K 14/39

(52) U.S. Cl. 514/8; 514/54; 536/123.12; 530/395

(57) ABSTRACT

The present invention provides an improved and more efficient method for the purification of Beta glucan, mannan and manno-protein complexes or immuno-potentiating substances, directly from yeast. In addition, the present invention provides methods for the production of Beta glucan, mannan and manno-proteins for use in animal feed to (1) prepare and enhance the immune system to fight infections more effectively and efficiently, (2) increase resistance to and decrease duration of bacterial and viral infections, (3) allow producers to reduce or remove “growth promotion antibiotics” in feed systems, thus producing more naturally grown animals, (4) reduce or prevent the negative growth response normally associated with administration of live vaccines to animals and (4) other associated health benefits such as in the case of swine of increased piglet litter size and survivability for piglets after weaning when Sows fed this novel Beta glucan extract.

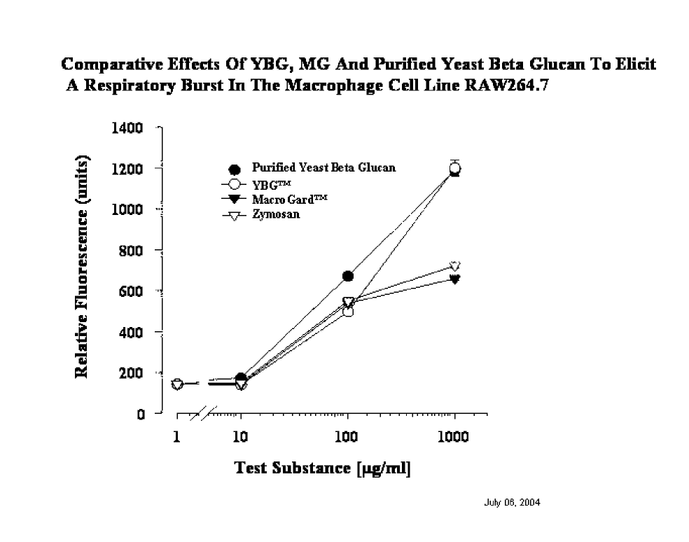

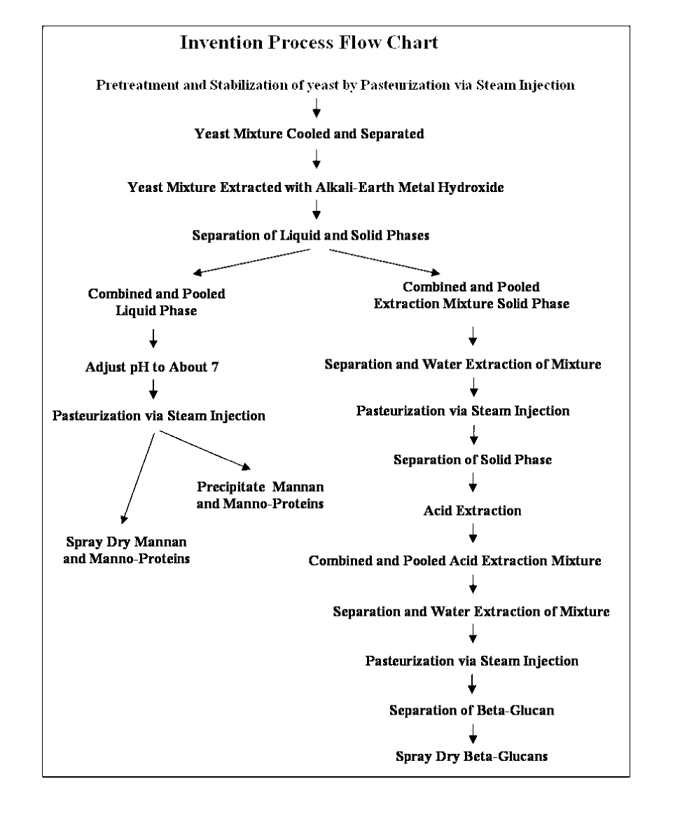

### FIELD OF INVENTION

The present invention relates to a process and use of poly-(1,3)-β-D-glucopyranosyl-(1,6)-β-D-glucopyranose (Beta-(1,3/1,6)-D-glucan), mannan and manno-protein complexes purified from yeast, fungi or bacterial cell walls which are capable of activating the innate immune system of animals and with other secondary health benefits such as an increase in the number of piglets born per sow and subsequent survivability. The invention also relates to the use of compositions comprised of purified Beta-(1,3/1,6)-D-glucan, mannan and manno-proteins that function to enable reduction and/or replacement of “growth promotion” antibiotics in animal feed to maintain animals, especially farmed animals, healthy and growing at an optimal rate. The invention optimizes equipment utilization, increases extraction efficiency resulting in higher yields, provides ecological benefits and decreases environmental impact as a result of reduced waste output as compared to other glucan extraction processes.

Definition of Immunobiotic: An agent (e.g., Beta glucan) or organism (e.g., lactic acid bacteria) that promotes health through broad-spectrum activation of intestinal, mucosal or systemic immunity.

### BACKGROUND OF THE INVENTION

Glucans are polysaccharides consisting of glucose subunits. Beta-(1,6) branched Beta-(1,3) glucan is a naturally occurring class of polysaccharides that can be extracted from Baker's yeast and other yeast species, mushrooms, plants and some bacterial, lichen and algal species (reviewed in Chemistry and Biology of (1,3)-β-Glucans, B. A. Stone and A. E. Clarke, 1992, La Trobe University Press, Australia). Beta-(1,6) branched (1,3) glucans have been shown to have immune enhancing and cholesterol-lowering capabilities. Mannans and manno-proteins are polysaccharide complexes that are naturally occurring and extracted from Baker's yeast and other yeast species, mushrooms, plants and some bacterial, lichen and algal species. Mannans and manno-protein complexes are beneficial in preventing the attachment of bacteria such as Escherichia coli to the gut lining, thus reducing the overall infection challenge in the body.

As a class of polysaccharides, Beta-(1,6) branched Beta-(1,3) glucans are composed of a main chain of glucose subunits linked together in (1,3) Beta glycosidic linkages and branches linked to the main chain by a (1,6) Beta glycosidic linkage. There are several different types of Beta glucans that vary in backbone composition, branching, type of monomers or substituents resulting in polysaccharides that have different physical and biological properties.

These glucans have been shown to activate the immune system of vertebrate as well as invertebrate organisms. One of the most powerful glucans that activates the immune system has been isolated from yeast. In the 1960's a crude extract of yeast, called Zymosan was shown to produce hyperplasia and stimulate the reticuloendothelial system, inactivate Complement Component C3, increase antibody formation, enhance survival following irradiation, increase resistance to bacterial infections, inhibit tumor development, and promote graft rejection. Subsequent studies have shown that the active component of Zymosan is Beta glucan.

Beta glucan from yeast activates the immune system by binding to a specific receptor on the cell membrane of macrophages (Czop and Kay, Isolation and Characterization of β-glucan Receptors on Human Mononuclear Phagocytes. J. Exp. Med. 173:1511-1520, 1991). The activated macrophages increase their phagocytic and bactericidal activities as well as the production of a number of cytokines, which in turn activate other components of the immune system (Di Luzio et al. in “The Macrophage in Neoplasia”, M. Fink, ed., 1976 Academic Press, New York, N.Y., pp 181-182).

U.S. Pat. No. 4,138,479 discloses a process for the preparation of a water-soluble Beta glucan from yeast with immunopotentiating activity.

U.S. Pat. No. 4,891,220 discloses methods and compositions to lower serum lipid levels using yeast Beta glucan and a cholesterol-regulating agent

U.S. Pat. No. 5,223,491 discloses a method for the preparation of water insoluble Beta glucan from yeast and its use for revitalizing skin by applying the glucan topically to the skin.

U.S. Pat. No. 5,397,773 discloses a composition using Beta glucan purified from yeast for use topically to prevent skin damage and repair skin damage caused by UV or solar radiation

U.S. Pat. No. 5,702,719 discloses methods and compositions using yeast Beta glucan for dermatological and nutritional uses

U.S. Pat. No. 5,817,643 discloses the preparation of soluble yeast glucan, which activates macrophages but does not stimulate macrophages to produce specific cytokines (e.g. IL-1 and TNF).

U.S. Pat. No. 6,444,448 discloses the preparation of insoluble yeast Beta glucan-mannan complexes by autolysis.

U.S. Pat. No. 6,214,337 discloses the preparation of an animal feed compromising yeast glucan when given orally, enhances animal growth.

Soluble yeast Beta glucan has been used in humans as an injectable to prevent and treat sepsis (Babineau et al., Randomized Phase I/II Trial of a Macrophage Specific Immunomodulator (PGG-glucan) in High-Risk Surgical Patients. Ann. Surgery 220:601-609, 1994). During the last few decades antibiotics have been the main drugs for the treatment of infections, including sepsis; however, during the last few years there has been the emergence of an alarming number of antibiotic-resistant bacteria, which are difficult to eradicate when they infect humans. The bacterial resistance has made infectious diseases the third leading cause of death in the United States, behind heart disease and cancer.

The emergence of antibiotic resistant bacteria has been linked to the increased and often unwarranted use of antibiotics in humans as well as to the widespread use of antibiotics as “growth promoters” in the feed of farmed animals.

Considered one of the most valuable antibiotic classes available to treat human infections, fluoroquinolones are used to treat a wide range of diseases, including the gastrointestinal illness caused by Campylobacter infection. However, poultry growers also use fluoroquinolones to keep chickens and turkeys from dying from Escherichia coli(E. coli) infection, a disease that they can pick up from their own droppings. While the drug may treat the E. coli infection in the poultry, other kinds of bacteria, such as Campylobacter, Salmonella or Clostridium perfringens may build up resistance to these drugs. People who consume poultry meat contaminated with fluoroquinolone-resistant Campylobacteror Salmonella are at risk of becoming infected with bacteria that current drugs cannot easily eradicate. Campylobacter is the most common bacterial cause of diarrheal illness in the United States, according to the Centers for Disease Control and Prevention. It's estimated to affect over 2 million persons every year, or 1 percent of the population. People infected with Campylobacter may be prescribed a fluoroquinolone, which may or may not work.

Nearly two million patients in the United States get an infection in the hospital each year. Of those patients, approximately 90,000 die each year as a result of their infection-up from 13,300 patient deaths in 1992. More than 70 percent of the bacteria that cause hospital-acquired infections are resistant to at least one of the antibiotics most commonly used to treat them. Persons infected with drug-resistant organisms are more likely to have longer hospital stays and require treatment with second or third choice antibiotics that may be less effective, more toxic and more expensive.

The relative ease with which antibiotic resistant bacteria can infect humans has been shown by the fact the antibiotic-resistant Salmonella strains have been found in meat sold in grocery stores (White et al., N. Engl. J. Med., 345:1147-1154, 2001) and by the fact that Enterococcus faecium bacteria found in grocery store meat will infect humans directly (McDonald et al., N. Engl. J. Med., 345: 1155-1160, 2001 and Sorensen et al., N. Engl. J. Med., 345: 1161-1166, 2001).

The concern with the widespread emergence of antibiotic-resistant bacteria has led the European Union to ban the use of antibiotics as “growth promoters” in animal feed. During the last few years in the United States a number of bills have been proposed that would ban or drastically reduce the use of antibiotics in agriculture. One such bill, The School Nutrition Safety Act, was presented to the US Congress on Sep. 5, 2003. This bill, H.R. 3022, was introduced “To protect children's health by ensuring that chickens and chicken products purchased for national school nutrition programs have not been fed or administered fluoroquinolone antibiotics”. This bill and others have not been voted into legislation yet, but because of the concern by scientists and various governmental organizations (e.g. CDC and the Department of Health), the use of antibiotics in agriculture will most likely be banned in the United States and most likely in most other countries. A ban on use of growth-promoting antibiotics would certainly increase the cost of farming animals, increase the cost of meats, and decrease meat supply unless a safe substitute for growth promotion antibiotics can be found.

Among the immunobiotic or immunoenhancing agents that have been investigated to enhance the activity of the immune system in humans and animals is a polysaccharide, Beta glucan, particularly the Beta glucan derived from the yeast Saccharomyces cerevisiae. Beta glucan is found in the cell walls of many yeast and cereal fibers. The biological activities of the glucans, which have been isolated from their natural state, demonstrate varied activity such as anti-infective and antibacterial as noted in Onderdonk A B, Cisneros R L. et al, “Anti-infective effect of poly-B-1,6 glucotriosyl-B-1,3-glucopyronose glucan in vivo” Infection and Immunity. 1992;60:1642-1647. Dr. Peter Mansell clearly identifies the anti-neoplastic benefits of a yeast Beta glucan in “Macrophage mediated destruction of human malignant cells in vivo”, Mansell P W, et al, Journal National Cancer Institute 1975;54:571-80. Stimulation of the immune response in humans and animals has become a priority in the prevention of infection and disease. Increasing resistance of pathogens by current antibiotics has added great significance to identifying the underlying cause of the faulty immune system. Identifying a natural, safe and effective immunomodulator has taken on tremendous interest. To this end, substantial work has been performed as evidenced in Abel, G. and Czop, J. K. “Stimulation of human monocyte Beta-glucan receptors by glucan particles induces production of TNF-alpha and IL-1 Beta, Int. Journal Immunopharmacolol, 14:1363-1373, 1992 and by Vetvicka, Vaclav et al, “Pilot Study: Orally-Administered Yeast B-1,3-glucan Prophylactically Protects Against Anthrax Infection and Cancer in Mice”, The Journal of the American Nutraceutical Association, Vol 5, No. 2, 2002. The benefits of Beta glucan through clinical use are presented by Ueno, H., “Beta-1,3-D-Glucan,” Japanese Journal Society Terminal Systemic Diseases, 6:151-154, 2000 as well as in U.S. Pat. No. 4,738,479. Yeast Beta glucan consists of straight chain and branched polymers. The straight chain structures are Beta-1,3-D-linked glucose polymers and Beta-1,6-D-linked glucose polymers. The branched polymers consist of a 1,3-Beta-D-linked backbone containing varying degrees of Beta-1,6-D branches. It is the quantity and distribution of these branches that influence the activity of the material. The amount of biological activity has a direct relationship to the varying degrees of purification.

There have been a number of reports regarding the purification and uses of Beta glucan from yeast, including its use in cosmetics (U.S. Pat. No. 5,223,491), to enhance resistance to diseases in aquatic animals (U.S. Pat. No. 5,401,727), and as a nutritional supplement for humans and animals (U.S. Pat. No. 5,576,0157, U.S. Pat. No. 6,214,337). The methods described in these patents use pure Baker's or Brewer's yeast or purified cell walls and various extraction procedures involving base and acid extractions at various temperatures. These procedures produce Beta-(1,3/1,6)-D-glucan with varying degrees of purity and varying levels of biological activity.

Our invention improves upon these methods by using spent Brewer's or Baker's yeast (Saccharomyces cerevisiae), fungi or bacterial cell walls to produce, during the same purification process, purified Beta-(1,3/1,6)-D-glucan, mannan and manno-protein complexes from yeast, which are capable of activating the innate immune system of animals and aid in preventing the adhesion of bacteria including E coli. to the intestinal wall of animals. Also disclosed is a method that, during this novel process, will protect and stabilize the Beta-(1,3/1,6)-D-glucan), mannan and manno-protein complexes from microbiological degradation to insure quality and biological activity while increasing process efficiencies and yields.

### SUMMARY OF THE INVENTION

The present invention utilizes Baker's or Brewer's yeast Saccharomyces cerevisiae but is not limited to them. Other yeast genus and fungi maybe employed. The yeast or fungi may be viable live or spent non-viable yeast. Sources for spent Baker's or Brewer's yeast as the starting material for the purification of Beta-(1,3/1,6)-D-glucan, mannan and manno-protein complexes, may be obtained directly from a brewery or other suitable vendor. The yeast may be in the form of a liquid, slurry or dry power. The present invention thus provides a method for inexpensive production of biologically and immunomodulatory active Beta-(1,3/1,6)-D-glucan, mannan and manno-protein complexes from yeast.

The method is very cost effective, such that the immunomodulatory Beta-(1,3/1,6)-D-glucan, mannan and manno-protein complexes produced can be used as a feed additive to enhance the immune competence of farmed animals at a cost competitive with current antibiotics. The mannans and manno-protein complexes add additional protection and reduce overall infection challenge by preventing pathogenic organisms such as Escherichia coli from attaching to the gut, thus the animal is less likely to develop an infection. Immune competence of animals treated with Beta-(1,3/1,6)-D-glucan produced by the present invention will increase resistance and will decrease the duration of both viral and bacterial infections by directly stimulating the innate immune system and secondarily antibody producing B cells. Furthermore, treatment of animals with this Beta glucan prior to administration of vaccines can boost the effectiveness of the vaccine while reducing or preventing the negative growth conditions usually attributed to the use of vaccines.

Furthermore, by recovering the mannans and manno-protein complexes the invention lowers the cost of manufacturing thus making an economical alternative to current antibiotics and previously disclosed methods. This inventive method recovers the mannans and manno-protein complexes from the alkali-earth metal liquid phase thus reducing sewered waste and making the process more ecologically friendly.

Accordingly, in one embodiment, the invention provides a method to stabilize and pretreat yeast (obtained from a brewery) by pasteurization via steam injection to a temperature of 100 degree C. for 15 to about 240 minutes. The mixture is cooled and separated into liquid and solid by a method such as centrifugation, filtration or other. The liquid phase is discarded and the solids are extracted by stirring with a 0.5 N to 5.0 N solution of an alkali-metal or alkali-earth metal hydroxide heated to a temperature of about 45 degree. C to about 120 degree C. for about 30 minutes to 240 minutes in a ratio of about 1:3 to 1:15 solids to liquid. The temperature is then increased to a range of about 95 degree C. to about 150 degree C. for about 15 min to about 240 min at a pressure of about 1 psi to about 25 psi. The extraction mixture is cooled and collected. The spent yeast and alkali-metal or alkali-earth metal hydroxide extraction is repeated 2 to 20 times and the resulting extraction mixture is combined and pooled with previous extractions. The pooled extraction mixture is mixed for 1 to 14 days. The solid and liquid phases are separated by centrifugation and collected. The yeast solids are water extracted at a ratio of 1:4 to about 1:20 with mixing for 15 minutes to about 4 hours at a temperature range of 20 degree C. to about 100 degree C. The resulting extraction mixture is cooled and collected. Water extractions of the yeast solids are repeated until all yeast solids have been separated. The resulting extraction mixture is combined and pooled with previous extractions. The combined and pooled extraction mixture is pasteurized by steam injection to a temperature of 100 degree C. for 15 to about 240 minutes to protect from microbiological degradation. The solids are separated and water extracted 2-3 times as before. The solids are separated from the mixture and subjected to extraction with an acid in a ratio of about 1:4 to about 1:20 solids to acid solution while being heated to a temperature from about 45 .degree. C. to about 120 degree C. for 15 minutes and maintained at 5 to 25 psi for about 2 hours. The solids are water extracted as before, collected and pasteurized. Solids separated from the acid treatment step will comprise at least 70% Beta-(1,3)/(1,6)-D-glucan by dry weight.

In another embodiment, the invention provides a method by which mannan and manno-proteins complexes are obtained from the separation of the liquid phase from the alkali-metal or alkali-earth metal hydroxide extraction and spent yeast. The liquid phase of the separation is collected and pH adjusted to 5.0-8.0 with an acid. The solution is pasteurized by steam injection to a temperature of 100 degree C. for 15 to about 120 minutes. The precipitated mannan and manno-proteins are collected and dried. Additional mannan and manno-proteins are precipitated at a ratio of about 1:0.25 by the addition of alcohol. The most preferred alcohol is ethanol but other maybe employed. The additional precipitated mannan and manno-protein complexes are collected and dried.

In another embodiment, the invention provides an additive to animal feed comprising Beta-(1,3)/(1,6)-D-glucan which may be used alone or in combination with mannan and manno-proteins prepared by this invention in an amount effective to enhance the innate immune system of animals, and reduce adhesion of pathogenic organisms in the gut. The most abundant source of protein in the world today is derived from the intensive farming of animals, primarily chickens, turkeys, swine and cattle and fish. The increasing world population and the increasing dependence on animal protein by developing countries is increasing the necessity for animal farming. Intensive animal farming involving increased stocking densities has been made possible by the widespread use of antibiotics to maintain animal health while maintaining the accumulation of muscle (meat protein) at a higher rate; commercially grown chickens using growth promotion antibiotics reach a net weight (0.5 to 2 Kg in 6 weeks). A pig will reach a net weight of about 75 Kg in 120 days.

During the last few years, it has become evident that the widespread use of antibiotics has led to the emergence of antibiotic resistant strains of bacteria that can infect humans and are difficult and expensive to treat. Because of the appearance of antibiotic resistant bacteria, the European Union has banned the addition of growth promotion antibiotics to animal feed. Many countries have followed and in North America a growing consumer awareness will most likely lead, in the near future, to a ban on the use of antibiotics similar to that in the European Union. Enhancement of the immune system of an animal will result in a heightened ability by the animal to combat infections making the addition of antibiotic to feed unnecessary.

### DETAILED DESCRIPTION OF THE INVENTION

The invention will now be described in detail by way of reference only to the following non-limiting examples.

### EXAMPLE 1

Purification Of Beta-(1,3)/(1,6)-D-glucan from spent yeast.

A 1.0 L sample of spent yeast (approximately 15% dry weight) was pasteurized by steam injection at a temperature of 100 degree C. for 20 minutes. The mixture was then separated by centrifugation, the liquid phase was discarded and the remaining solids were re-suspended in 1:5 volumes water (volume/volume) with mixing for 15 minutes. The mixture was then separated by centrifugation, the liquid was discarded and the solids were suspended in 10 volumes (weight/volume) of 1.5 M NaOH. The mixture was then heated with stirring to 80 degree C. for 45 minutes and autoclaved for 30 minutes. The mixture is cooled and left to stand with mixing at ambient temperature. The NaOH spent yeast extraction was repeated two additional times and the mixture was combined with the first extraction mixture. The pooled NaOH mixture was left to stand with mixing at ambient temperature for 24 hours. The solids and liquid phase were separated by centrifugation and collected. The solids were water extracted with mixing at 20 degree C. and separated by centrifugation. The solids were collected and the liquid phase discarded. The solids were water extracted as before. After the completion of the second water extraction the solution was pasteurized by steam injection to a temperature of 100 degree C. for 20 minutes. The pasteurized solution was cooled and left to stand at ambient temperature with mixing for 24 hours. The solids were separated by centrifugation and collected. The liquid phase was discarded. The solids are subjected to 3% acetic acid extraction in a ratio of 1:10 solids to acid at a temperature 80 degree C. for 1 hour with mixing. Solids separated from the acid treatment are washed with water, pasteurized, separated, collected and spray dried. The composition of the spray-dried material is shown in Table 1.

TABLE 1
Composition of purified Beta-(1,3)/(1,6)-D-glucan

| Component | Quantity |
|---|---|
| Carbohydrate | >70% |
| Glucose/mannose | 56:1 |
| Lipid | <25 |
| Protein | <3 |
| Biological Activity | >40 mg Bb |
| (Alternative | released/mg |
| Complement) | |

### EXAMPLE 2

Separation of Mannan and Manno-Protein Complexes from the Alkali-Earth Metal Extraction Liquid Phase.

The liquid phase from the NaOH extractions of the separation from Example 1 was collected. The pH of the mixture was adjusted to 7.0 with HCl. The solution was pasteurized by steam injection to a temperature of 100 degree C. for 20 minutes. The precipitated mannans and manno-proteins were collected by centrifugation. The liquid phase of the separation was treated with ethanol and the precipitates collected. The precipitated mannans and manno-proteins were combined and dried. The composition of the dried material is shown in Table 2.

TABLE 2
Composition of Mannans and Manno-Protein Complexes

| Component | Quantity |
|---|---|
| Carbohydrate | >35% |
| Lipid | <5% |
| Protein | >15% |
| Sulfated Ash | <20% |

### EXAMPLE 3

Drawing 1 demonstrates the comparative effects of several commercially available yeast Beta glucans, including YBG, which was produced by the inventive method.

### EXAMPLE 4

The use of Beta-(1,3)/(1,6)-D-glucan as an additive to the feed of pigs as substitute for antibiotics.

Influence of Beta-(1,3)/(1,6)-D-glucan on the growth efficiency and immune response of pigs vaccinated with a PRRSv attenuated vaccine and a saline control is shown in Table 3a. This study, conducted on weaned pigs, compares: growth, health, and response to vaccination, when incremental doses of YBG were included in their diets for 4 weeks after weaning. The results indicated that YBG was able to increase the antibody response to vaccination when included at 80 mg/kg in the diet. The vaccinated animals, when fed YBG, grew faster than their counterparts who received only the vaccine. The conclusion was that YBG was able to boost the immune response of pigs and improve the growth rate during an immune system challenge.

TABLE 3a

| Average Daily Growth Rate | g/day | S.E. | |
|---|---|---|---|
| control | 436 | 21 | a |
| Beta glucan 40 mg/kg | 425 | 25 | a |
| Beta glucan 80 mg/kg | 390 | 25 | a |
| PRRSv Control | 363 | 22 | C |
| PRRSv @ PBG 40 mg/kg | 400 | 21 | a, c |
| PRRSv@ PBG 80 mg/kg | 428 | 22 | a |
| PRRSv @ PBG 120 mg/kg | 362 | 21 | c |
| Feed Conversion | F:G | S.E. | |
| Control | 1.626 | 0.044 | a |
| Beta glucan 40 mg/kg | 1.618 | 0.054 | a |
| Beta glucan 80 mg/kg | 1.773 | 0.053 | b |
| Beta glucan 120 mg/kg | 1.607 | 0.047 | a |
| PRRSv Control | 1.735 | 0.046 | b |
| PRRSv @ PBG 40 mg/kg | 1.655 | 0.045 | a, b |
| PRRSv@ PBG 80 mg/kg | 1.716 | 0.046 | a, b |
| PRRSv @ PBG 120 mg/kg | 1.653 | 0.045 | a, b |

PRRS Antibody Response (Elisa)

| | S/P ratio | |
|---|---|---|
| | Relative | |
| Treatment | Immune response) | SE |
| Control | 1.49 | 0.18 a |
| PRRSv @ PBG 40 mg/kg | 1.62 | 0.18 a |
| PRRSv@ PBG 80 mg/kg | 2.20 | 0.18 b |
| PRRSv @ PBG 120 mg/kg | 1.26 | 0.22 a |

It appears purified Beta-Glucan may have a significant impact on weaned pigs. This study had the following findings:
Two doses of oil adjuvant and live virus injections may negatively impact the growth rate and feed conversion of weaned pigs,
Purified Beta Glucan may reduce some of the vaccine associated growth reduction at a dose of 80 mg/kg of feed,
Purified Beta Glucan may increase the antibody response to live virus vaccination at a dose of 80 mg/kg of feed

The results were published at the following two conferences:
a. Proceedings of the Annual Meeting of the American Association of Swine Veterinarians, March 2004, Des Moines, Iowa.
b. 18^{th} Congress of the International Pig Veterinary Society, June 2004, Hamburg, Germany.

The effect of Beta-(1,3)/(1,6)-D-glucan on the gestating sow and the impact on the number of piglet born alive and number weaned is shown in Table 3b. When the purified beta glucan produced by this invention is administered to gestating sows at 80 mg/kg the number of piglets born per sow increased as well as the number of piglets (10.8% increase over control) that survived past the weaning stage (5.4% increase over control). Results for beta glucan compared to the control are significant (p<0.05). This translates into significant savings for the swine producers.

TABLE 3b

| Treatment | Average Piglets Born Alive | Average Piglet Weaned |
|---|---|---|
| Control | 10.35 | 9.27 |
| Beta Glucan | 11.47 | 9.96 |
| Vitamin premix | 11.15 | 9.32 |

### EXAMPLE 5

Effect of Beta-(1,3)/(1,6)-D-glucan on the growth of chickens.

These studies were performed on farms in Nova Scotia, Canada. A total of 18500 chickens housed on the bottom tier of three different houses were fed a diet containing 20-40 grams of Beta glucan/1000 Kg of feed while 18500 chickens housed on the top tier of the same houses were fed a diet containing the antibiotic Stafac. All feed in both groups was supplemented with the coccidiostat Coban. At the end of the six weeks, performance was assessed using the following criteria: mortality, weight, feed conversion, condemnation rate. The results of this experiment summarized in Table 4 below show comparable growth parameters in chickens fed on the two feed regimens, indicating that it is feasible to farm chickens without antibiotics.

TABLE 4
Effect of Beta (1,3)/(1,6)-D-glucan on growth of Chickens as
compared to “growth promotion antibiotics”.

| Criteria | Beta Glucan + Coban | Stafac + Coban |
|---|---|---|
| Total Number of | 25,431 | 25,564 |
| Birds places | | |
| Mortality (%) | 1.63% | 1.81% |
| Weight (kg) | 2.03 | 2.02 |
| Age (days) | 40.42 | 40.42 |
| Average Daily | 50.59 | 50.69 |
| Gain (g/day) | | |
| Producer | 0.65% | 0.65% |
| Condemnation % | | |
| Feed Conversion² | 1.82% | 1.78% |

NB.
Table summarizes a total of four (4) independent commercial trials of approximately 6500 chicks/trial/treatment
¹Stafac and Coban was obtained/supplied by ACA-Coop Ltd
²Feed conversions are based on the ratio of food consumed to mass of bird. Typical commercial ratios range from 1.5-1.9.

### EXAMPLE 6

Effect of Beta-(1,3)/(1,6)-D-glucan on immune parameters in chickens.

To determine the effectiveness of Beta (1,3)/(1,6)-D-glucan as immune enhancer, lymphocytes isolated from the blood of chicken fed a diet containing an average of 25 grams of purified Beta glucan and lymphocytes isolated from chickens fed a diet containing antibiotics were analyzed for proliferative capacity in the presence of the following blastogenic substances, CA, PHA, PMA+lonomycin, LPS, and PWM. Table 5 shows that in animals fed a diet containing growth promotion antibiotics, the response of lymphocytes to these various substances was heterogeneous with a significantly low stimulation of immune cells. The response to CA, PWM, and LPS was either not significantly different from the control and in some animals the response was significantly less than in the control (e.g., response to PHA in animals Nos. 1, 5, 7, and 20; response to PMA in animal 13; response to PWM in animals 13 and 20, response to LPS in animal 13.). Thus animals fed with only growth promotion antibiotics have an un-activated immune system that would potentially be more susceptible to bacterial infections that the antibiotic does not treat. Furthermore, antibiotics have no effects on treating or resisting viral infections.

TABLE 5
YBG Treated Chickens
Increased Immune Stimulation

| | C1 | C4 | C6 | C9 | C14 | C15 | C16 | C18 | C21 | C24 | AVG. |
|---|---|---|---|---|---|---|---|---|---|---|---|
| Control | 100 | 100 | 100 | 100 | 100 | 100 | 100 | 100 | 100 | 100 | 100 |
| Con A | 163 | 133 | 134 | 114 | 116 | 113 | 107 | 120 | 107 | 98 | 121 |
| PHA | 173 | 95 | 112 | 121 | 108 | 122 | 127 | 100 | 127 | 119 | 120 |
| PWM | 147 | 128 | 149 | 121 | 112 | 101 | 116 | 106 | 113 | 164 | 126 |
| LPS + DxS | 139 | 148 | 159 | 118 | 148 | 123 | 147 | 120 | 116 | 152 | 137 |
| PMA + Iono | 173 | 129 | 143 | 100 | 130 | 125 | 126 | 120 | 108 | 160 | 131 |

Increase in cell number upon stimulation, expressed as percentage of unstimulated matching controls for chickens fed a feed containing the immuno-modulator beta glucan (YBG) derived from yeast Saccharomyes cerevisiae.

On the other hand, Table 6 shows that animals fed a diet containing Beta glucan, the response of lymphocytes to the various substances was significantly more positive since the proliferating of lymphocytes was more than the unstimulated matching controls. The response was especially elevated following PMA and LPS stimulation. These data are indicative of an enhanced immune competence and increased resistance to both bacterial and viral infections in animals fed purified Beta glucan.

TABLE 6
Antibiotic Treated Chickens
No Change or Decrease in Immune Stimulation

| | C2 | C3 | C5 | C7 | C8 | C10 | C11 | C13 | C17 | C19 | C20 | C22 | AVG. |
|---|---|---|---|---|---|---|---|---|---|---|---|---|---|
| Control | 100 | 100 | 100 | 100 | 100 | 100 | 100 | 100 | 100 | 100 | 100 | 100 | 100 |
| Con A | 128 | 95 | 95 | 95 | 97 | 87 | 110 | 92 | 100 | 103 | 97 | 92 | 99 |
| PHA | 72 | 86 | 56 | 69 | 85 | 94 | 91 | 94 | 83 | 106 | 74 | 81 | 83 |
| PWM | 81 | 88 | 106 | 85 | 89 | 73 | 84 | 66 | 92 | 106 | 86 | 104 | 88 |
| LPS + DxS | 100 | 126 | 165 | 100 | 100 | 103 | 139 | 63 | 118 | 133 | 83 | 102 | 111 |
| PMA + Iono | 106 | 103 | 147 | 136 | 97 | 96 | 178 | 53 | 102 | 117 | 89 | 104 | 111 |

Increase in cell number upon stimulation, expressed as percentage of unstimulated matching controls for chickens fed a feed containing a growth promotion antibiotic.

### EXAMPLE 7

Growth Comparison between YBG and Antibiotic Fed Broilers.

Trials held at the ACA Cooperative, Ltd., a commercial broiler and turkey Co-op, resulted in equivalent growth performance of Broiler Chickens in the absence of antibiotics. The response in turkeys was an increase from the typical 85%-90% “Grade A” birds to >90% “Grade A” birds.

Although the invention has been described with reference to the presently preferred embodiment, it should be understood that various modifications could be made without departing from the spirit of the invention. Accordingly, the invention is limited only by the following claims.

1. A method using spent Brewer's or Bakers yeast, fungi, or bacterial cell walls to produce a Beta-(1,3/1,6)-D-glucan, mannan and manno-protein complexes, said method comprising:
a) stabilization and pretreatment of yeast by pasteurization using steam injection to a temperature of about 100 degree C. for 15 to about 30 minutes;
b) cooling the stabilized, pretreated yeast and separating into liquid and solid phases;
c) extraction of the solid phase with stirring with a 0.5 N to 5.0 N solution of an alkali-metal or alkali-earth metal hydroxide heated to a temperature of about 45 degree C. to about 80 degree C. for about 30 minutes in a ratio of about 1:5 to 1:15 solids to liquid;
d) increasing the temperature to 95 degree C. to about 150 degree C. for 15 min to about 120 min at a pressure of 1 psi to about 25 psi;
e) extracting with water the solid phase separated from solid-liquid phase of d) at a ratio of 1:4 to about 1:20 with mixing for 15 minutes to about 2.5 hours at a temperature range of 20 degree C. to about 100 degree C.;
f) pasteurization by steam injection to a temperature of about 100 degree C. for 15 to about 30 minutes;
g) extraction of the solid phase from f) with an acid in a ratio of about 1:4 to about 1:20 solids to acid solution while being heated to a temperature from about 45 degree C. to about 120 degree C. for 15 minutes to about 2 hours;
h) extraction with water the solid phase separated from solid-liquid phase of g) at a ratio of 1:4 to about 1:20 with mixing for 15 minutes to about 2.5 hours at a temperature range of 20 degree C. to about 100 degree C.;
i) pasteurization by steam injection to a temperature of about 100 degree C. for 15 to about 30 minutes;
j) Solids separated from i) will comprise at least 70% Beta (1,3)/(1,6)-D-glucan by dry weight.

2. The method of claim 1, wherein the yeast is pasteurized using steam injection to a temperature of 100 degree C. for 20 minutes.

3. The method of claim 1, wherein the separation of b) is by centrifugation.

4. The method of claim 1, wherein the alkali-metal or alkali-earth metal hydroxide is sodium hydroxide.

5. The method of claim 4, wherein the sodium hydroxide is 1.5 M.

6. The method of claim 1, wherein the temperature of c and d) was 80.degree. C. for 45 minutes and 121 degree C. for 30 minutes.

7. The method of claim 1, wherein a) through d) is repeated 3 times.

8. The method of claim 7, wherein the mixture is collected and combined.

9. The method of claim 8, wherein the mixture is left to stand with mixing at ambient temperature.

10. The method of claim 9, wherein the duration was about 24 hours.

11. The method of claim 1, wherein the separation in e) was by centrifugation.

12. The method of claim 1, wherein the water extraction in e) was at a ratio of about 1:5.

13. The method of claim 1, wherein the water extraction in e) was at a temperature of about 20 degree C.

14. The method of claim 1, wherein the water extraction in e) duration was about 30 minutes.

15. The method of claim 1, wherein the water extraction in f) is pasteurized using steam injection to a temperature of 100 degree C. for 20 minutes.

16. The method of claim 1, wherein the separation in f) was by centrifugation.

17. The method of claim 1, wherein the acid in g) was acetic acid.

18. The method of claim 17, the acetic acid was about 3%.

19. The method of claim 1, wherein in g) the duration was about 1 hour.

20. The method of claim 1, wherein g) through h) is repeated about 3 times.

21. The method of claim 1, wherein the separation in h) was by centrifugation.

22. The method of claim 1, wherein the water extraction in h) was at a ratio of about 1:5.

23. The method of claim 1, wherein the water extraction in h) was at a temperature of about 20 degree C.

24. The method of claim 1, wherein the water extraction in h) duration was about 30 minutes.

25. The method of claim 1, wherein the water extraction in i) is pasteurized using steam injection to a temperature of 100 degree C. for 20 minutes.

26. The method of claim 25, wherein was separated by centrifugation.

27. The method of claim 26, wherein was spray dried.

28. The method of claim 11, wherein the mannan and manno-protein complexes are obtained from the liquid phase;
k) the liquid phase of the separation is collected;
l) the solution's pH is adjusted to 5.0-8.0 with an acid;
m) the solution is pasteurization by steam injection to a temperature of about 100 degree C. for 15 to about 30 minutes;
n) the mannan and manno-protein complexes are precipitated by alcohol or spray dried.

29. The method of claim 28, wherein the acid in k) was hydrochloric acid.

30. The method of claim 28, wherein the pH in k) was about 7.

31. The method of claim 28, wherein in 1) is pasteurized using steam injection to a temperature of 100 degree C. for 20 minutes.

32. The method of claim 28, wherein the solids from n) will comprise at least 30% mannan and manno-protein complexes.

33. The method of claim 28, wherein the solids from n) will comprise at least 15% protein.

34. A method that protects and stabilized the (1,3)/(1,6)-D-glucan from micro-biological degradation by the method of claim 1.

35. Preparation of an animal feed comprising Beta (1,3)/(1,6)-D-glucan prepared by the method of claim 1 in an amount effective for enhancing immuno-competence of the animals.

36. The animal feed of claim 35, wherein the effective amount is a concentration of the Beta (1,3)/(1,6)-D-glucan in the range from about 5 ppm to about 500 ppm of the feed.

37. The animal feed of claim 36, wherein the animal fed the feed containing Beta glucan is in the poultry family.

38. The animal feed of claim 36, wherein the animal fed a feed containing Beta glucan is in the family of swine.

39. The animal feed of claim 36, wherein the animal fed a feed containing Beta glucan is in the family of horses.

40. The animal feed of claim 36, wherein the animal fed a feed containing Beta glucan is in the family of cattle.

41. The animal feed of claim 36, wherein the animal fed the feed containing Beta glucan is in the family of crustacean.

42. A method for enhancing the immune competence of poultry, said method comprising adding an effective amount of Beta (1,3)/(1,6)-D-glucan produced by the methods in claim 1 to poultry feed.

43. The method of claim 42, wherein the effective amount is between 5 ppm and 500 ppm of feed.

44. The method of claim 43, wherein the effective amount is 20 ppm.

45. A method for enhancing the immune competence of swine, said method comprising adding an effective amount of Beta (1,3)/(1,6)-D-glucan produced by the methods in claim 1 to swine feed.

46. The method of claim 45, wherein the effective amount is from 25 ppm to 300 ppm of feed and more preferably 100 ppm.

47. The method of claim 45, wherein the effective amount is 80 ppm.

48. A method for enhancing the immune competence of horses, said method comprising adding an effective amount of Beta (1,3)/(1,6)-D-glucan produced by the methods in claim 1 to horse feed.

49. The method of claim 48, wherein the effective amount is from 25 ppm to 300 ppm of feed and more preferably 100 ppm.

50. The method of claim 48, wherein the effective amount is 60 ppm.

51. A method for enhancing the immune competence of shrimp, said method comprising adding an effective amount of Beta (1,3)/(1,6)-D-glucan produced by the methods in claim 1 to shrimp feed.

52. The method of claim 51, wherein the effective amount is between 35 ppm and 300 ppm.

53. The method of claim 52, wherein the effective amount is 80 ppm.

54. A method for enhancing the antibody formation in swine said method comprising adding an effective amount of Beta (1,3)/(1,6)-D-glucan produced by the methods in claim 1 to feed.

55. A method for enhancing the antibody formation as well as reducing the negative growth responses usually associated with administering a vaccine.

56. Preparation of an animal feed comprising Beta (1,3)/(1,6)-D-glucan prepared by the method of claim 1 in an amount effective for enhancing immuno-competence of the animals combined with mannans and mannoproteins prepared by method claim 28, an amount sufficient to inhibit bacterial adhesion to the intestinal walls of livestock and laboratory animals.

57. The method of claim 26, wherein the Beta glucan is added to the feed at 10 ppm to 200 ppm of feed and the mannans are added at 500 ppm to 4 ppm of feed.
